(19) United States
(12) Patent Application Publication
(10) Pub. No.: US 2026/0069285 A1
(43) Pub. Date: Mar. 12, 2026

(54) ROTARY INSTRUMENT FOR DENTAL IMPLANTOLOGY OR ENDODONTICS, COMPRISING A FLEXIBLE PART

(71) Applicant: REED DENTAL TECHNOLOGIES SÀRL, Lausanne (CH)

(72) Inventor: Frédéric WEHRLI, Lausanne (CH)

(21) Appl. No.: 18/863,974

(22) PCT Filed: Mar. 7, 2023
(86) PCT No.: PCT/IB2023/052132

§ 371 Date: Nov. 7, 2024

(30) Foreign Application Priority Data
May 13, 2022 (WO) PCT/IB2022/054490

(51) Int. Cl. A61B 17/16; A61C 8/00

(52) U.S. Cl. CPC A61B 17/1615; A61B 17/1631; A61B 17/1673; A61C 8/0089; A61B 17/1655

(57) ABSTRACT

The invention relates to a rotary instrument (1) for dental implantology or endodontics, comprising at least one proximal part (10) configured to be connected to a rotational drive means, and a distal part (20), the shape of which is determined by the use of the instrument; the instrument comprising flexible means that make the instrument (1) flexible.

### CORRESPONDING APPLICATION

The present application claims priority from the earlier PCT international application No. PCT/IB2022/054490 filed on May 13, 2022 in the name of Frédéric Wehrli, the contents of this earlier PCT application being incorporated by reference in its entirety into the present application.

### FIELD OF THE INVENTION

The present invention relates to dental implantology and endodontics and more precisely to the instruments used for this purpose.

### PRIOR ART

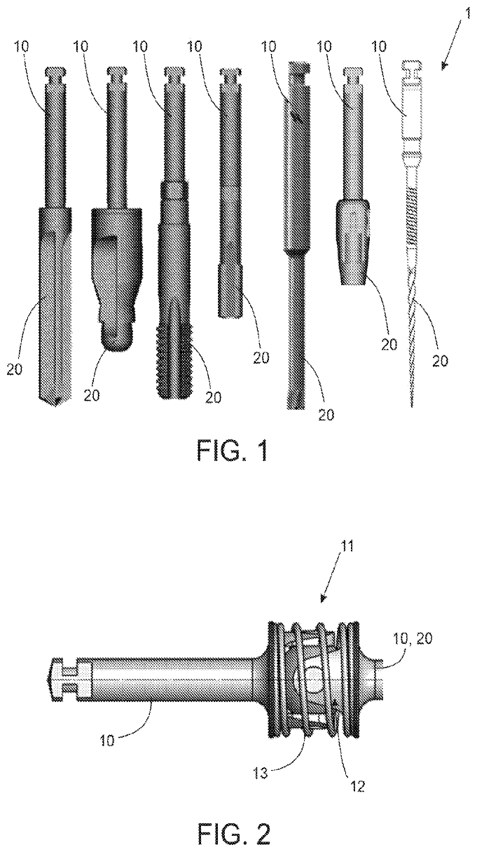

Rotary instruments for dental implantology and endodontics are surgical instruments which, when used in a well-defined sequence, allow dentists to perform dental treatments using a motorized device. In implantology, these instruments are used to prepare and place a dental implant in the bone of a patient's jaw. In endodontics, the corresponding rotary instruments allow the removal of the dental pulp from a tooth whose nerve is damaged (root treatment). Rotary instruments for implantology and endodontics comprise in particular two functionally distinct parts as illustrated in FIG. 1, namely:
a proximal part (also known as the “standard part”), for example in the form of a rod, provided with a standardized end, for example of type ISO 1797-1, allowing the instrument to be connected to a motor by way of a contra-angle in order for it to be driven in rotation,
a distal part (also known as the “specific part”) whose design (for example its shape and/or size) derives from the main function of the instrument in question (drilling, boring, tapping, screwing, filing, etc.).

In FIG. 1, examples of rotary instruments for implantology or endodontics with functionally distinct parts (standard or proximal part 10 at the top, specific or distal part 20 at the bottom) are illustrated, from left to right: a drill, a reamer, a tap, an implant holder, a screwdriver for a prosthetic screw, an extension piece and an endodontic file. Of course, these are examples of instruments, and the use of the invention is not limited to these examples. As will be understood from the description below, the invention also applies to other equivalent instruments.

These instruments are, by their design, totally rigid, or at least totally rigid in their proximal part, which is desirable for drills or reamers, but which can become a major drawback for taps, screwdrivers for prosthetic screws, endodontic files and especially for implant holders (instruments for gripping an implant and inserting it into the bone). In fact, for the latter, a certain flexibility in bending can be an undeniable advantage in practice. Such flexibility would make it possible in particular to avoid hyper-guidance, often a source of considerable stresses within the instrument itself, but also within the bone and/or the implant. In some cases, these stresses can even lead to bone damage (necrosis) or even fracture of the implant.

In practice, there are therefore contradictory demands in respect of such instruments: on the one hand, rigidity for certain operations and, on the other hand, the need to have more flexible instruments of use for other operations, while maintaining standardization of said instruments.

### GENERAL DESCRIPTION OF THE INVENTION

An object of the present invention is to improve the known instruments and to overcome the aforementioned problems.

Another object of the present invention is to propose standard instruments which can nevertheless be adapted to the wishes of the users, in particular the dentists or surgeons concerned.

The invention relates to a rotary instrument for dental implantology or endodontics, comprising:
a proximal part configured to be connected to a drive means, such as a rotary motor, by way of a contra-angle (or other equivalent means),
a distal part whose shape is specific to the use of the instrument.

The invention is characterized in particular in that the instrument comprises means allowing flexibility of said instrument.

In some embodiments, the flexible means are permanently flexible. In other embodiments, the flexible means may have at least two states: one rigid, the other flexible. Other states are possible, such as semi-rigid (or semi-flexible).

In some embodiments, the flexible means are located on the proximal part of the instrument.

In some embodiments, the flexible means are located, for example added, between the proximal part and the distal part of the instrument.

In other embodiments, the flexible means are located, for example added, between the proximal part and means for connecting the instrument to the drive means, such as a motor.

In some embodiments, the flexible means comprise an articulation.

In some embodiments, the proximal part is formed of a flexible component, or comprises a flexible component, said flexible component being sufficiently torsionally rigid for the intended use of the instrument. This material is for example Nitinol (alloy of nickel and titanium) or another equivalent material (e.g. metal, metal alloy, etc.).

In some embodiments, the articulation comprises a universal joint formed of at least two forks and of a central yoke of substantially spherical shape.

In some embodiments, the universal joint comprises a pin and a pair of half pins, and the central yoke is traversed by two perpendicular passages dimensioned to receive the pin and the pair of half pins.

In some embodiments, the central yoke comprises two perpendicular grooves, and the forks comprise guides dimensioned so as to slide in said grooves.

In some embodiments, the flexible means comprise a spiral spring and/or a wire-shaped spring.

In some embodiments, the instrument, e.g. its proximal part, comprises a plurality of flexible means.

In some embodiments, the instrument may be a drill, a reamer, a tap, an implant holder, a screwdriver, a file, an adapter or an extension piece that can be connected to one or more other instruments.

Other embodiments and features of the invention are described in the claims and in the following description.

### DETAILED DESCRIPTION OF THE INVENTION

The invention is illustrated in greater detail in the remainder of the description, in particular by way of several examples and embodiments, all of which must be considered as illustrative and non-limiting.

FIG. 1 illustrates examples of rotary instruments for implantology or endodontics.

FIG. 2 illustrates an embodiment of the invention in the assembled state.

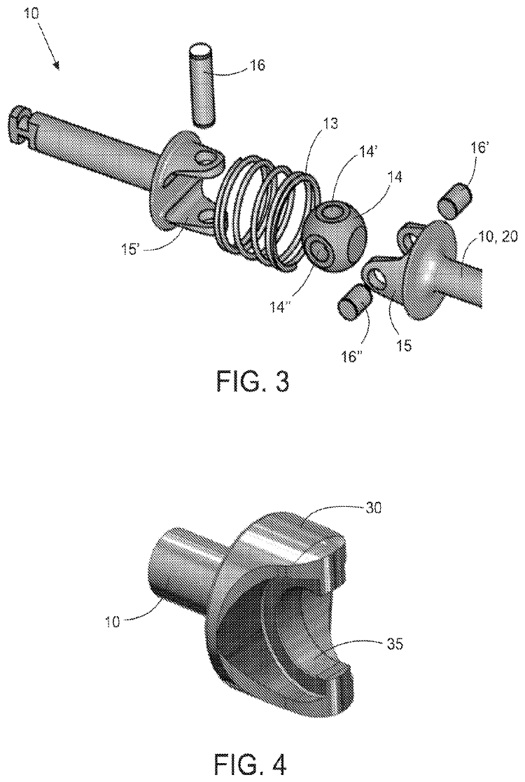

FIG. 3 illustrates the embodiment from FIG. 2 in exploded form.

FIGS. 4 to 7 illustrate another embodiment of the invention.

FIG. 8 illustrates another embodiment of the invention.

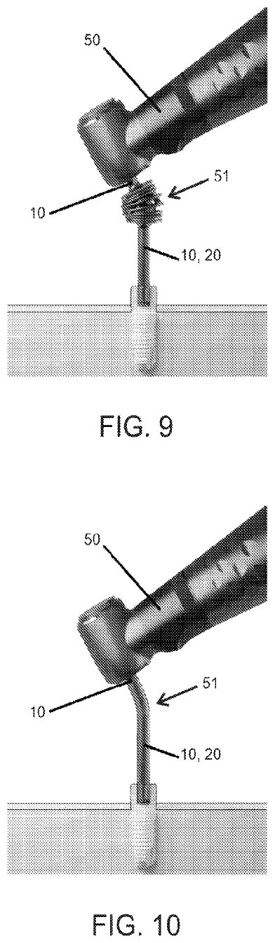

FIGS. 9 and 10 illustrate the use of embodiments of the invention for an implant holder (as a non-limiting example).

One embodiment is illustrated in FIG. 2. In this embodiment, the proximal part 10 of the instrument 1 comprises flexible means with an articulation 11 comprising a universal joint 12 coupled to a flexion spring 13.

As is shown in FIG. 3, the universal joint 12 from FIG. 2 comprises a central yoke 14 to which two forks 15, 15′ are joined with the aid of a pin 16 and two half-pins 16′, 16″. The pins 16, 16′, 16″ are inserted into passages 14′, 14″ in the yoke 14 in order to form the articulation. Fastening means are of course provided so that the pins remain in place once inserted.

The axes of the pin 16 and of the two half-pins 16′, 16″ are preferably perpendicular in order to avoid any oscillations during the rotation of the instrument. This is desirable because the oscillations can induce stresses in the instrument 1, the bone or the implant, which would be precisely the opposite of what is sought by the invention.

The universal joint 11 may be located on the proximal part 10, or between the proximal part 10 and distal part 20, which allows the articulation to be completely free in flexion while possessing excellent torsional rigidity, a property necessary for good transmission of the torque in the instrument.

For its part, the flexion spring 13 imparts semi-rigidity in flexion to the entire articulation, ensuring that the instrument 1 has a certain strength. The spring 13 also allows the instrument 1 to fold reversibly.

FIGS. 4 to 7 illustrate another embodiment of the invention using forks and a grooved sphere.

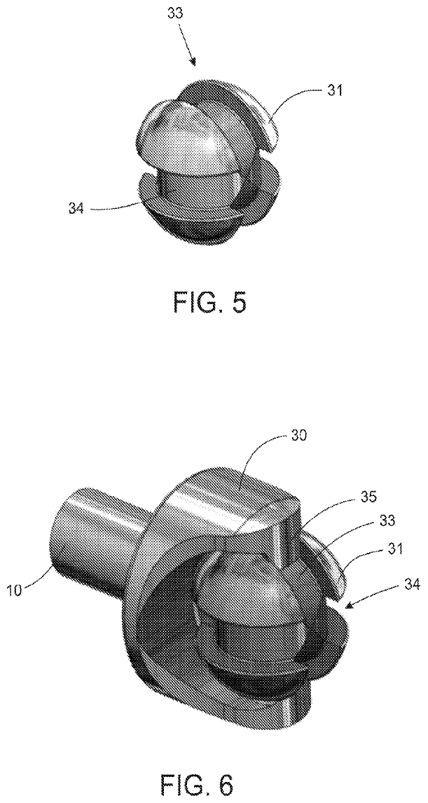

In FIG. 4, a fork 30 is illustrated which is fixed to the proximal part 10 of the instrument. FIG. 5 illustrates a grooved sphere 31 on which the fork 30 will engage (for example by forcing), as is illustrated in FIG. 6 by its guide 35 engaged in the groove 33.

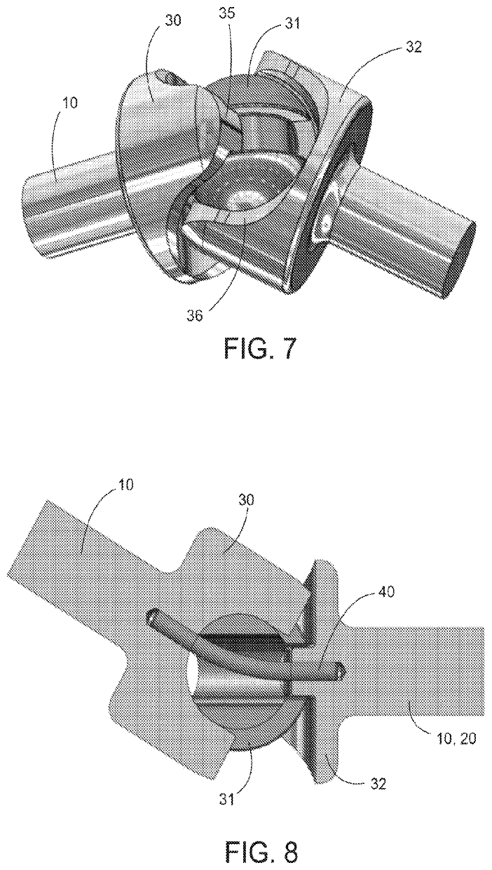

FIG. 7 illustrates the joining of a second fork 32 to the sphere 31 (for example by forcing, the guide 36 sliding in the groove 34) facing the fork 30 and placed at 90° so as to form an articulation similar to the universal joint of FIGS. 2 and 3.

In this embodiment, it is possible, in a variant, to add a spring 13, as in the embodiment of FIG. 2.

For this purpose, shoulders are preferably added in order to retain the spring in position around the articulation.

FIG. 8 illustrates another embodiment of the invention. In this embodiment, a wire 40, for example made of NiTi or steel (or another equivalent material), is inserted through the forks 30, 32 and the sphere 31 in order to ensure flexural rigidity.

The flexible means of FIGS. 4 to 8 may be located on the proximal part 10 of the instrument, or between the proximal part 10 and distal part 20 of the instrument, or between the proximal part 10 and the means of connection to the motor 50 (as means of driving the instrument), see FIG. 9.

The embodiment in which the proximal part 10 is formed of a flexible component, or comprises a flexible component, said flexible component being sufficiently rigid in torsion for the intended use of the instrument, is illustrated, for example, in FIG. 10 where the proximal part 10 is formed partially or completely of this flexible component.

In the case of an implant holder 51, by virtue of the flexible means illustrated in the embodiments above, the function of the instrument (to insert the implant into the bone) can be ensured even when the available space is restricted as illustrated in FIG. 9.

In this case, the instrument holder (contra-angle 50) is held at an angle (for example up to 30°) by the surgeon. As is illustrated in FIGS. 9 and 10, by virtue of the flexible articulation, the implant holder 51, connected to the contra-angle, can perform its function even when the inter-occlusal height is small.

It goes without saying that the invention is not limited to the aforementioned examples and use, and that the invention and its advantages can be used in various applications.

Embodiments have been described in order to provide a comprehensive understanding of the principles of structure, function, manufacture and use of the systems and methods described in the present application. One or more of these embodiments are illustrated in the accompanying drawings. The systems and methods specifically described in the present application and illustrated in the accompanying drawings are non-limiting embodiments, and the scope of the present invention is not defined solely by the claims. The features illustrated or described in relation to one embodiment may be combined with the features of other embodiments. Such modifications and variants are intended to be included within the scope of the present invention. A number of problems with the conventional methods and systems are noted herein, and the methods and systems described herein may solve one or more of these problems. Moreover, while this invention has been described in conjunction with a certain number of embodiments, the alternatives, modifications, equivalents and variants which are within the spirit and scope of the present invention are also covered by the present application.

1. A rotary instrument for dental implantology or endodontics, comprising at least
a proximal part configured to be connected to a rotational drive means,
a distal part whose shape is determined by the use of the instrument, characterized in that the instrument comprises flexible means allowing flexibility of said instrument.

2. The instrument as claimed in claim 1, characterized in that the flexible means have two states, a flexible state and a rigid state.

3. The instrument as claimed in claim 1, characterized in that the flexible means are located on the proximal part of the instrument.

4. The instrument as claimed in claim 1, characterized in that the flexible means are located between the proximal part and the distal part of the instrument.

5. The instrument as claimed in claim 1, characterized in that the flexible means are located between the proximal part and the means for connection to the drive means.

6. The instrument as claimed in claim 1, characterized in that the flexible means comprise an articulation.

7. The instrument as claimed in claim 6, characterized in that the articulation comprises a universal joint formed of at least two forks and a central yoke of substantially spherical shape.

8. The instrument as claimed in claim 7, characterized in that the universal joint comprises a pin and a pair of half-pins, and in that the central yoke is traversed by two perpendicular passages dimensioned so as to receive the pin and the pair of half-pins.

9. The instrument as claimed in claim 1, characterized in that the central yoke comprises two perpendicular grooves and in that the forks comprise guides dimensioned so as to slide in said grooves.

10. The instrument as claimed in claim 1, characterized in that the flexible means comprise a spiral spring.

11. The instrument as claimed in claim 1, characterized in that the flexible means comprise a wire-shaped spring.

12. The instrument as claimed in claim 1, wherein the proximal part comprises a plurality of flexible means.

13. The instrument as claimed in claim 1, said instrument being a drill, a reamer, a tap, an implant holder, a screwdriver, a file, an adapter or an extension piece that can be connected to one or more other instruments.
